(19) United States
(12) Patent Application Publication
(10) Pub. No.: US 2008/0260669 A1
(43) Pub. Date: Oct. 23, 2008

(54) Aromachemicals

(75) Inventor: Luca Turin, London (GB)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Flexitral, Inc., Chantilly, VA (US)

(21) Appl. No.: 11/738,919

(22) Filed: Apr. 23, 2007

(51) Int. Cl. A61K 8/34; A23G 4/06; A23L 1/22; A61P 43/00; A61Q 15/00; C07D 333/02; C11D 3/50; B65D 85/00; A61Q 13/00; A23L 2/56; A61K 31/381

(52) U.S. Cl. 424/65; 426/106; 426/3; 426/531; 426/535; 426/590; 510/103; 512/11; 514/438; 549/79

(57) ABSTRACT

A compound of formula (I):

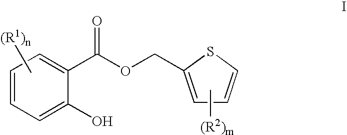

wherein R¹ and R² represent, independently, linear or branched C₁₋₄ alkyl, n=0 to 4 and m=0 to 3. Mixtures of these compounds, methods for their preparation, their use as perfume materials for application to a variety of substrates and their use in flavouring and in articles of manufacture is also provided.

### BACKGROUND OF THE INVENTION

1. Field of the Invention

The present invention relates generally to the field of flavors and fragrances. More particularly, the present invention relates to new aromachemicals that are believed to provide perfumes and other articles with properties and advantages not shared by many known aromachemicals. These aromachemicals find utility in any and all applications requiring flavors and fragrances. The invention also provides mixtures of these aromachemicals, methods for their preparation and their use as perfume materials for application in a variety of substrates and their use in flavoring and articles of manufacture and compositions including the aromachemicals.

2. Description of the Related Art

There are a large number and variety of known flavors and fragrances used as ingredients in perfumes and in a varied range of other products. However, many aromachemicals include double bonds and/or other reactive groups that are potentially susceptible to reaction and may result in a limited useful lifetime. Further, many essential oil fragrances have recently been determined to cause allergic reactions, and it is becoming increasingly difficult to bring products containing such fragrances to market. There is also a demand for new flavours and fragrances that have novel or improved fragrance profiles and/or other properties that make them particularly useful for use as fragrances and/or flavours.

The listing or discussion of a prior-published document in this specification should not necessarily be taken as an acknowledgement that the document is part of the state of the art or common general knowledge.

It is an object of the invention to provide new aromachemical compounds. It is also an object of the invention to provide a method for producing these aromachemical compounds.

### SUMMARY OF THE INVENTION

The present invention provides compounds of formula (I):

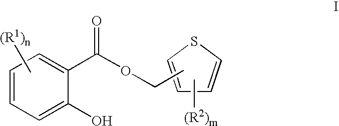

wherein R¹ and R² represent, independently, linear or branched C₁₋₄ alkyl, n=0 to 4 and m=0 to 3.

By linear or branched C₁₋₄ alkyl we mean methyl, ethyl, n-propyl, i-propyl, n-butyl, i-butyl, s-butyl or t-butyl. If n>1, each R¹ group may be the same or different. If m>1, each R² group also may be the same or different. Preferably, n=0 and/or m=0.

The compounds of formula (I) are referred to hereinafter as “the compounds of the invention” or “the aromachemicals of the invention”.

A preferred group of compounds of the invention are those in which the methanol salicylate ester group is attached to the 2-position of the thiophene ring. Specific examples of such compounds include the compound of formula (I′), namely 2-thiopene methanol salicylate ester.

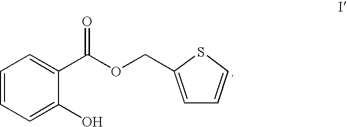

This compound possesses balsamic, sweet, floral and mild balsamic notes and has excellent blending capabilities. The compound of formula (I′) may be used as the foundation for heavy florals such as ylang, gardenia, etc. This compound is also believed to be a replacement for the EU allergen benzyl salicylate. In this regard, it is a more powerful aromachemical than benzyl salicylate by a factor of 3-5 and therefore less likely to be allergenic. The other compounds of the invention are alaso believed to exhibit one or more of these advantageous features.

The present invention provides a method for the preparation of a compound of formula (I) comprising reacting a compound of formula

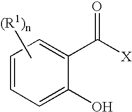

with a compound of formula

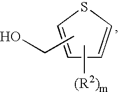

wherein R¹ and R² represent, independently, linear or branched C₁₋₄ alkyl, n=0 to 4, m=0 to 3 and X is selected from the group consisting of —OMe, —OH and Cl. If n>1, each R¹ group may be the same or different. If m>1, each R² group also may be the same or different. Prefereably, the CH₂OH group of the compound of formula

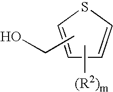

is attached to the 2-position of the thiophene ring. Preferably, n=0 and/or m=0. Preferably X=—OMe. In which case, the reaction is carried out in the presence of an acid or base catalyst (e.g. sodium methoxide).

Compounds of the invention may be isolated from their reaction mixtures using conventional techniques including, for example, recrystallisation, filtration, column chromatography and distillation.

The present invention provides for the use of a compound or a mixture of compounds of the invention as a flavor and/or fragrance. For example, the compound of formula (I′) described above is particularly suitable for use as a flavour and/or fragrance.

The present invention also provides a substrate treated with a compound of the invention and a corresponding method for treating the substrate to impart flavourant/fragrance releasing characteristics thereto. The compounds of formula (I′) described above is particularly suitable for such substrates/methods.

The present invention also provides compositions, products, preparations or articles having aroma, fragrance or odour releasing characteristics containing a compound or mixture of compounds of the invention as described above, such as the compound of formula (I′), optionally in admixture with other perfuming ingredients, solvents or adjuvants of current use in the art.

The present invention provides a method to confer, improve, enhance or modify the taste or flavor property of a composition, product, preparation or article which comprises adding thereto a flavor effective amount of a compound or mixture of compounds of the invention as described above, such as the compound of formula (I′).

The present invention also provides a method to confer, improve, enhance or modify the aroma, fragrance or odor characteristics of compositions, products, preparations or articles which comprises adding thereto an aroma, fragrance or odor effective amount of a compound or mixture of compounds of the invention as described above, such as the compounds of formula (I′) as defined above, is also provided.

The present invention provides an article of manufacture comprising packaging material and an aroma, odour, fragrance, taste or flavour enhancing agent contained within the packaging material. The agent is a compound or mixture of compounds of the invention and is effective for the enhancement of the aroma, odour, fragrance, taste or flavour of a composition, preparation, product or article to which it is added. The packaging material comprises a label which indicates that the agent can be used for enhancing aroma, odour, fragrance, taste or flavour.

### DETAILED DESCRIPTION OF THE INVENTION

The compounds of the invention can be included in virtually any article of manufacture that can include fragrance or flavorant compounds. Examples include hypochlorite (bleach) compositions, detergents, flavorings and fragrances, beverages, including alcoholic beverages, and the like. The compounds of the invention can be used in applications like soaps, shampoos, denture cleanser tablets, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they may be used in other current uses in perfumery, for example the perfuming of soaps and shower gels, hygiene or hair-care products, as well as of body deodorants, air fresheners and cosmetic preparations, and even in fine perfumery, such as in perfumes and colognes.

The compounds of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

The compounds of the invention can be used as perfuming ingredients, as single compounds or as mixtures thereof. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through their general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the compounds of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of the invention are typically present at concentrations between about 0.01 and about 30%, or even more, by weight of these compounds relative to the weight of the composition, product or article in which they are incorporated. It will be appreciated that the amount by weight of a compound of the invention in a particular composition or product will depend on the nature of the composition. For example, a washing powder will typically contain less than 1% by weight of a compound of the invention while a fine fragrance may contain more than 20% by weight of a compound of the invention.

The compounds may be used in detergents such as those containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These aspects are described in more detail below.

In addition to the compounds of the invention, the compositions described herein may include a detersive surfactant and optionally, one or more additional detergent ingredients, including materials for assisting or enhancing cleaning performance, treatment of the substrate to be cleaned, or to modify the aesthetics of the detergent composition (e.g. perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random C₁₀₋₂₀ alkyl sulfates (“AS”), and the like. Preferred compositions incorporating only synthetic detergents have a detergent level of from about 0.5% to 50%. Compositions containing soap preferably comprise from about 10% to about 90% soap.

The compositions described herein can contain other ingredients such as enzymes, bleaches, fabric softening agents, dye transfer inhibitors, suds suppressors, and chelating agents, all well known within the art.

The compounds of the invention can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and/or fruit juice concentrates.

Artificial colorants that may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the compounds of the invention herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the compounds of the invention can also be prepared. The compounds of the invention can be incorporated into conventional foodstuffs using techniques well known to those of skill in the art. Alternatively, the compounds can be incorporated within polymeric particles, which can, in turn, be dispersed within and/or over a surface of an orally-deliverable matrix material, which is usually a solid or semi-solid substrate. When used in chewable compositions, the compounds of the invention can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

Flavored compositions of the invention may include an orally-deliverable matrix material; a plurality of water insoluble polymeric particles dispersed in the orally-deliverable matrix material, where the polymeric particles individually define networks of internal pores and are non-degradable in the digestive tract; and one or more compounds of the invention entrapped within the internal pore networks. The compounds of the invention are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound or a mixture of compounds of the invention to a desired flavor concentration. In one method for producing such gum based products a blade mixer is heated to about 110° F., the gum base is preheated so that it is softened, and the gum base is then added to the mixer and allowed to mix for approximately 30 seconds. The compound or compounds of the invention are then added to the mixer and mixed for a suitable amount of time. The gum can be then removed from the mixer and rolled to stick thickness on waxed paper while warm.

The compounds of the invention may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles that contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy)lower alkyl 2-alkeneoate and a polyethylenically unsaturated crosslinking agent. These materials have continuous slow release properties, i.e. they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The present invention is illustrated by the following non-limiting example.

### Synthesis of 2-thiophene methanol salicylate ester

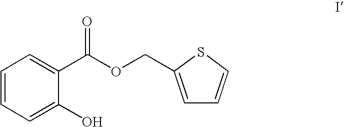

In a 100 ml round bottom flask equipped with a distillation apparatus, methyl salicylate (33 g, 0.22 mol), 2-methanol-thiophene (25 g, 0.22 mol) and sodium methoxide (0.5 g, 0.02 mol) were heated at 95° C. during 12 hours. The reaction was followed by ¹H NMR. The mixture was taken into dichloromethane, filtered and after evaporation of the solvent, the crude product was purified by column chromatography on silica gel eluting with ethyl acetate/hexane (1%/99%) to give the desired product and methyl salicylate which was removed under distillation (122° C. at 15 mbar). 2-thiophene methanol salicylate ester crystallised as a white solid (13.8 g, 59 mmol).

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A compound of formula (I): 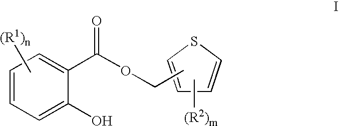
wherein R¹ and R² represent, independently, linear or branched C₁₋₄ alkyl, n=0 to 4 and m=0 to 3.

2. A compound according to claim 1 wherein the methanol salicylate ester group is attached to the 2-position of the thiophene ring.

3. A compound according to claim 1 wherein n=0.

4. A compound according to claim 1 wherein m=0.

5. A compound according to claim 1 wherein n=0 and m=0.

6. A method for the preparation of a compound of formula (I) 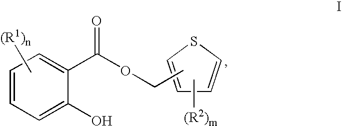
comprising reacting a compound of formula 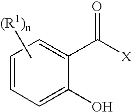
with a compound of formula 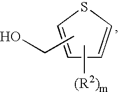
wherein R¹ and R² represent, independently, linear or branched C₁₋₄ alkyl, n=0 to 4, m=0 to 3 and X is selected from the group consisting of —OMe, —OH and Cl.

7. A method according to claim 6 wherein the CH₂OH group is attached to the 2-position of the thiophene ring.

8. A method according to claim 6 wherein n=0.

9. A method according to claim 6 wherein m=0.

10. A method according to claim 6 wherein X=—OMe.

11. A method according to claim 6 wherein n=0, m=0 and X=—OMe.

12. A composition comprising a flavour or fragrance enhancing amount of a compound or a mixture of compounds as defined in claim 1.

13. A substrate treated with a compound or a mixture of compounds as defined in claim 1.

14. A method for treating a substrate to impart flavourant/fragrance releasing characteristics thereto comprising treating the substrate with a flavour or fragrance enhancing amount of a compound or a mixture of compounds as defined in claim 1.

15. A composition, product, preparation or article having aroma, fragrance or odour releasing characteristics containing a compound or mixture of compounds as defined in claim 1 in admixture with other perfuming ingredients, solvents, or adjuvants of current use in the art.

16. A composition, product, preparation or article according to claim 15 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

17. A composition, product, preparation or article according to claim 16, wherein the compound or mixture of compounds is in admixture with at least one detergent ingredient and/or at least one bleach ingredient and/or at least one disinfectant ingredient.

18. A composition, product, preparation or article according to claim 15 in the form of a body odorant, deodorant or antiperspirant wherein the compound or mixture of compounds is in admixture with other body odorant, deodorant or antiperspirant ingredients, solvents and/or adjuvants.

19. A beverage composition, product, preparation or article comprising a compound or mixture of compounds according to claim 1 and one or more additional beverage ingredient.

20. A flavouring composition, product, preparation or article comprising a compound or mixture of compounds according to claim 1 and a solvent, adjuvant and/or another flavouring ingredient.

21. A food composition, product, preparation or article comprising a compound or mixture of compounds according to claim 1 and a solvent, adjuvant and/or another food ingredient.

22. A chewing gum composition, product, preparation or article comprising a compound or mixture of compounds according to claim 1 and a solvent, adjuvant and/or another chewing gum ingredient.

23. A pharmaceutical composition, product, preparation or article comprising a compound or mixture of compounds according to claim 1 and a pharmaceutically active ingredient.

24. An orally-deliverable matrix comprising a compound or mixture of compounds according to claim 1 and at least one matrix material.

25. A method to confer, improve, enhance or modify a taste or flavour property of a composition, product, preparation or article which comprises adding thereto a flavour effective amount of a compound or mixture of compounds as defined in claim 1.

26. A method according to claim 25, wherein said composition, product, preparation or article is in the form of a beverage, a flavouring, a food, a chewing gum, a pharmaceutical or an orally deliverable matrix.

27. A method to confer, improve, enhance or modify an aroma, fragrance or odour characteristics of a composition, product, preparation or article which comprises adding thereto an aroma, fragrance or odour effective amount of a compound or mixture of compounds as defined in claim 1.

28. A method according to claim 27 wherein said composition, product, preparation or article is in the form of a perfume, a body odorant, deodorant or antiperspirant, a detergent, a bleach product or a disinfectant.

29. An article of manufacture comprising packaging material and an aroma, odour, fragrance, taste or flavour enhancing agent contained within the packaging material, wherein the agent is in an amount effective for the enhancement of the aroma, odour, fragrance, taste or flavour of a composition, preparation, product or article to which it is added, and wherein the packaging material comprises a label which indicates that the agent can be used for enhancing aroma, odour, fragrance, taste or flavour, and wherein the agent is a compound or mixture of compounds as defined in claim 1.
